(19) United States
(12) Patent Application Publication
(10) Pub. No.: US 2003/0165919 A1
(43) Pub. Date: Sep. 4, 2003

(54) Method for the analysis of exogenic and endogenic cell activiation based on measuring the aggregation of receptors

(76) Inventors: Gerd Schmitz, Regensburg (DE); Alexandra Gotz, Regensburg (DE)

Correspondence Address: Albert P Halluin, Howrey Siom Arnold & White, Box 34, 301 Ravenswood Avenue, Menlo Park, CA, 94025, US

(21) Appl. No.: 10/220,757

(22) PCT Filed: Mar. 5, 2001
(86) PCT No.: PCT/DE01/00817

(30) Foreign Application Priority Data
Mar. 3, 2000 (DE) 100 10 474.6
Jun. 5, 2000 (DE) 100 27 308.4

(51) Int. Cl. C12Q 1/68; G01N 33/53; G01N 33/567

(52) U.S. Cl. 435/6; 435/7.2

(57) ABSTRACT

The present invention relates to a method for analysing exogenous and endogenous cell activation where the assembly of receptors in a receptor cluster comprising CD14 is measured. Preferably, this takes place by measuring the energy transfer between the individual receptors, measurement by means of fluorescence resonance energy transfer (FRET) being preferred. The present invention further relates to a method for diagnosing systemic inflammations such as sepsis, arteriitis or autoimmune diseases or an arteriosclerotic or inflammatory disease of the coronary arteries (CAD, for example angina pectoris, cardiac infarction or coronary scleroses) or of the cerebral arteries (such as a stroke) or a precursor of any of these diseases which is based on the measurement of the assembly of receptors in a receptor cluster comprising CD14, as well as the use of compounds preventing clustering of CD14 to treat these diseases.

The present invention relates to a method for analysing exogenous and endogenous cell activation comprising measuring the assembly of receptors in a receptor cluster comprising CD14. Preferably, this takes place by measuring the energy transfer between the individual receptors, measurement by means of fluorescence resonance energy transfer (FRET) being preferred. The present invention further relates to a method for diagnosing systemic inflammations such as sepsis, arteriitis or autoimmune diseases or an arteriosclerotic or inflammatory disease of the coronary arteries (CAD, for example angina pectoris, cardiac infarction or coronary scleroses) or of the cerebral arteries (such as a stroke) or a precursor of any of these diseases which is based on the measurement of the assembly of receptors in a receptor cluster comprising CD14, as well as the use of compounds preventing clustering of CD14 to treat these diseases.

CD14, an important surface receptor on monocytes plays an important role in inflammatory reactions as a response to bacterial pathogenic agents. More recent data indicate that the expression of CD14 is changed when 2 systemic inflammation is present. In addition, the expression of CD14 is reduced in a HMG-CoA reductase inhibitor therapy and the expansion of a more strongly differentiated monocyte subpopulation with high CD14 expression and of the Fcγ receptor IIIa (Fcγ-RIIIa, CD 16a) correlates with an atherogenic lipoprotein profile, which indicates a regulatory function of this receptor both in cases of a systemic inflammation and diseases of the coronary arteries.

Even though lipopolysaccharide (LPS) is the main ligand of CD14, more recent data indicate that other substances also bind to CD14, including, but not limited to lipoteichoic acid, anionic phospholipids, soluble peptidoglycan, muramyl dipeptide, polymannuoronic acid, lipoarabino mannane and other products of microbial origin. Recently, it was shown that stress-inducible members of the heat shock protein 70 (HSP70) family induce cytokine production in monocytes by a CD14 mediated signal transduction path. It is also known that HSP70 binds a ceramide derivative, sulfogalactosyl ceramide (SG-ccr).

Since CD14 is a glycosyl phospbatidyl inositol-anchored receptor (GPI-R) without transmembrane domain, it was assumed that additional membrane proteins contribute to the signal transduction, because CD14 on its own is unable to transduct signals. There are strong indications that one (or more) members of the “toll-like” receptor (TLR) family, especially TLR4 and TLR2 contribute to the LPS-induced signal transduction. Genetic and species-specific differences in the recognition of lipid A indicate that TLR4 is the essential molecule giving signals to LPS. However, it has not been possible to date to prove a direct physical interaction of LPS or CD14 with TLRs. Like CD14, various other GPI receptors such as the urokinase plasminogen activator receptor (uPA-R, CD87) and Decay Accelerating Factor (DAF, CD55) and Protectin (CD59) also associate with detergent-insoluble, glycolipid-enriched domains (DIGs). It was shown that these DIGs are membrane domains rich in cholesterol and sphingolipid which are also called “rafts”. After receptor aggregation, smaller “raft” domains result in the formation of larger receptor clusters or larger raft domains which associate with signal-emitting compounds on the cytosol side.

The central role of CD14 in the activation of monocytes indicates that additional CD14 ligands exist. In addition, very little is known about the potential of the different CD14 ligands with regard to the induction of specific receptor complexes or clusters, for example on The surface of human monocytes, because, to date, no reliable and uncomplicated processes are available which permit the measurement of such a formation of receptor clusters or, indirectly, of the cell activation they induce.

Therefore, the present invention is based on the technical problem to provide means permitting the analysis of exogenous or endogenous cell activation, for example cell activation resulting in the release of mediators promoting inflammation or inducing atherosclerosis.

This technical problem is solved by providing the embodiments characterised in the patent claims. It was found that cell activation may be determined by measuring the assembly of receptors in a receptor cluster comprising CD14. This was shown through various measuring methods, particular emphasis being placed on the measurement of the energy transfer between the receptors, preferably by fluorescence resonance energy transfer (FRET). We measured the clustering of the glycosyl phosphatidyl inositol-anchored endotoxin receptor CD14 which, as discussed above, plays an important role in the inflammatory response in monocytes as a reaction to lipopolysaccharide (LPS). It was also found that ceramide, a component of atherogenic lipoproteins, acts as an extracellular ligand of CD14. By a comparison of the effects of LPS and ceramide, receptor/ligand interactions based on the clustering of CD14 with co-receptors were investigated. It was shown that both LPS and ceramides induced the co-assembly of CD14, complement receptor 3 (CD11b/CD18), CD36 and DAF (CD55). It was interesting to note that only LPS induced the co-assembly with the “toll-like” receptor 4 (TLR 4), FcγIIIa CD16a) and the integrin-associated protein (IAP) CD81, while ceramide induced clustering with CD47 (another IAP). This indicates that there are two different cellular paths which correlate with the assembly of congenital receptor complexes in “rafts”. In case of sepsis, the binding of LPS to CD14 is accompanied by a release of inflammation-promoting mediators and the binding of ceramide to CD14 correlates to atherosclerosis. Ceramide seems to be a modulator of the immune function in atherosclerosis.

Therefore, the subject matter of the present invention is a method for analysing exogenous and endogenous cell activation comprising the measurement of the assembly of receptors in a receptor cluster comprising CD14.

The term “exogenous cell activation” designates the induction of membrane-associated signal transduction cascades by exogenous stimuli (stimuli originating from outside the body).

The term “endogenous cell activation” designates the induction of membrane-associated signal transduction cascades by endogenous stimuli (stimuli originating from inside the body).

The term “measurement of the assembly of receptors in a receptor cluster comprising CD14” comprises every method which may be used to determine the assembly, i.e. the vicinity, of receptors in such a receptor cluster. Examples of such methods are scanning nearfield optical microscopy (SNOM), atomic force microscopy (AFM), chemical cross-linking, co-capping, and single dye tracing farfield video rates fluorescence microscopy. Measurement of energy transfer between the receptors, preferably measurement by means of FRET also is advantageous, and a measurement combining FRET with flow cytometric energy transfer, FCDT, is especially preferred. Measurement of energy transfer should be understood to mean that it is not the energy transfer between the assembling receptors that is measured directly, but that between the antibodies binding to the different receptors, said energy transfer being indicative of a co-assembly of the relevant receptors, i.e. a vicinity to each other of about 2 to 10 nm, and thus of cell activation.

In a preferred embodiment of the method of the invention, measurement of the assembly of receptors in a receptor cluster comprising CD14 is carried out by measuring the energy transfer between the receptors, preferably by means of FRET, more preferably by combining FRET with FCDT. A method according to the invention where an energy transfer efficiency or an energy transfer parameter (Etₚ) of >5% is indicative of endogenous or exogenous cell activation is especially preferred. This value may be measured in accordance with the following example 1 (I).

In another preferred embodiment of the method of the invention, the receptor cluster comprising CD14 also includes LPS-associated proteins, e.g. TLR-4, integrin-associated proteins e.g. CD81 and CD47, G-protein receptors, complement regulating proteins, e.g. CD11b, CD18 and CD55, Fcγ receptors, e.g. CD16a, CD32 and CD64, and/or scavenger receptors, e.g. CD36. In an even more preferred embodiment of the method of the invention, the energy transfer between an antibody against one of the above proteins (receptors), especially CD11b, and an anti-CD14 antibody is measured.

The present invention also relates to a method for diagnosing systemic inflammations such as sepsis, arteriitis or an autoimmune disease, or an arteriosclerotic or inflammatory disease of the coronary arteries (CAD such as angina pectoris, cardiac infarction or coronary scleroses) or cerebral arteries (such as stroke), respectively, or a precursor of one of these diseases, characterised in that the measurement of the assembly of receptors on monocytes of a patient sample (e.g. fresh lithium-heparin whole blood, about 1 ml) is carried out in accordance with the method of the invention described above, an energy transfer, preferably an energy transfer efficiency or an energy transfer parameter (Etₚ) of >5% being indicative for a systemic inflammations such as sepsis, arteriitis or autoimmune diseases, or an arteriosclerotic or inflammatory disease of the coronary arteries (CAD. such as angina pectoris, cardiac infarction or coronary sclerosis) or of the cerebral arteries (such a stroke) or a precursor of these diseases.

Since the above diseases may be caused by clustering of the receptors described above, e.g. systemic inflammations such as sepsis, arteriitis or autoimmune diseases, or an arteriosclerotic or inflammatory disease of the coronary arteries (CAD such as angina pectoris, cardiac infarction or coronary scleroses) or cerebral arteries (such as stroke), therapy or prevention of these diseases may be carried out by preventing such clustering.

Therefore, the present invention also relates to the use of a compound neutralising exogenous inflammation-promoting molecules such as LPS, lipoteichoic acid, ceramides and associated proteins, e.g. SG-ccr/HSP70, endogenous inflammation-mediating molecules and/or phosphollipids, e.g. phosphatidyl inositol or phosphotidyl ethanol amine, for the treatment of systemic inflammations such as sepsis, arteriitis or autoimmune diseases, or an arteriosclerotic or inflammatory disease of the coronary arteries (CAD such as angina pectoris, cardiac infarction or coronary scleroses) or cerebral arteries (such as stroke) or a precursor of these diseases. The same applies for compounds neutralising LPS-associated receptors such as CD14 and TLR-4, complement-regulating proteins such as CD11b, CD18 and CD55, Fcγ-R such as CD16a, CD32 and CD64, integrin-associated proteins such as CD81 and CD47 and/or scavenger receptors e.g. CD36. The term “neutralising compound” includes any compound which may bind to these ligands or receptors in such a manner and modify them in such a manner that the clustering of CD14 with the other receptors is blocked completely or to a substantial extent. Identification of such substances and the examination of their effectiveness may be made by routine methods, for example by measuring whether they prevent an energy transfer. Preferably, the ceramide-neutralising compound is a CD24:1 neutralising compound. For example, the effect may be due to the fact that the binding of the CD14 ligands to CD14 is blocked and clustering therefore prevented. This may be achieved by using antibodies which either bind to the receptor ligand itself or the receptor (CD14 and/or one of the receptors discussed above) in such a manner that binding of the ligand to its receptor is blocked. Alternatively, this may be achieved by administration of a competitive analogous substance [such as an antagonist or the extracellular (soluble) portion of the receptor], the binding of the biologically active ligand to the natural receptor being reduced or eliminated altogether by the (preferred) binding of the ligand to the receptor analogue or the (preferred) binding of the antagonist to the natural receptor.

In a preferred embodiment the neutralising compound is a specifically binding antibody, preferably a monoclonal antibody or a fragment thereof.

Methods for recovering such antibodies are known to the person skilled in the art and, as far as polyclonal antibodies are concerned, for example, involve the use of the above-mentioned ligands or receptors, e.g. CD14, or a fragment thereof as an immunogen for immunising suitable animals and recovering serum. Methods for preparing monoclonal antibodies are also known to the person skilled in the art. For this purpose, cell hybrids from antibody-producing cells and bone marrow tumour cells are prepared and cloned. After that, a clone producing an antibody specific for the desired immunogen is selected. This antibody is then prepared. Examples of cells producing antibodies are spleen cells, lymph node cells. B-lymphocytes, etc. Examples of animals that may be immunised for this purpose are mice, rats, horses, goats, and rabbits. The myeloma cells may be obtained from mice, rats, humans or other sources. Cell fusion, for example, may be carried out by the well known method of K\check{o}hler and Milstein. The hybridomae obtained by cell fusion are selected through the immunogen according to the enzyme-antibody method or a similar method. Clones, for example, are obtained by the borderline dilution process. The clones obtained are implanted intraperitoneally into BALB/c mice. After 10 to 14 days, the ascites of the mouse is removed and the monoclonal antibody purified by known processes (such as ammonium sulfate fractionation, PEG fractionation, ion exchange chromatography, gel chromatography or affinity chromatography). The antibody thus recovered may be used either directly or a fragment thereof may be used. In the present context, the term “fragment” includes all components of the monoclonal antibody (e.g. Fab, Fv or single chain Fv fragments) which have the same epitope specificity as the complete antibody.

In a particularly preferred embodiment, the above-mentioned monoclonal antibody is an antibody derived from an animal (e.g. a mouse), a humanised antibody or a chimeric antibody or a fragment thereof. Chimers. antibodies resembling human antibodies or humanised antibodies, have a reduced potential antigenicity, but their affinity vis-á-vis their target is not decreased. The preparation of chimeric and humanised antibodies or of antibodies resembling human antibodies has been extensively described in literature. Humanised immunoglobulins have variable areas in their basic structures which are substantially derived from an human immunoglobulin (designated acceptor immunoglobulin), and the complementarity of the determining areas is essentially derived from a non-human immunoglobulin (e.g. from the mouse) and is designated donor immunoglobulin. If present, the constant area(s) also is/are substantially derived from a human immunoglobulin. When administered to human patients, the humanised (and human) antibodies of the invention have a number of advantages over mouse antibodies or other species: (a) the human immune system should not identify the basic structure or the constant area of the humanised antibody as strange; therefore, the antibody response against such an injected antibody should be less pronounced than that against a completely strange mouse antibody or a partially strange chimeric antibody; (b) since the effector range of the humanised antibody is human, it will probably have improved interaction with other parts of the human immune system; and (c) injected humanised antibodies have a half life substantially equivalent to that of natural human antibodies, which permits the administration of smaller and less frequent doses as compared to antibodies of other species.

In a particularly preferred embodiment the neutralising compound is a soluble form of an LPS-associated receptor, e.g. of CD14 and TLR-4, a complement-regulating protein, e.g. of CD11b, CD18 and CD55, of Fcγ-R, e.g. of CD16a, CD32 and CD64, an integrin-associated protein, e.g. of CD81 and CD47, a scavenger receptor, e.g. CD36 and/or a G-protein receptor.

Optionally, the neutralising compound described above is administered in combination with a pharmaceutically compatible excipient. Suitable excipients and the formulation of such drugs are known to the person skilled in the art. Suitable examples of such excipients include phosphate-buffered saline, water, emulsions such as oil/water emulsions, wetting agents, sterile solutions, etc. The proper dose is determined by the physician in charge of treatment and depends on several factors, e.g. age, sex, the weight of the patient, type and stage of the disease, the route of administration, etc.

### SHORT DESCRIPTION OF THE FIGURES

FIG. 1:

Ligand-dependent Change of the Conformation of the CD14/CD11b Complex

a. Human monocytes were incubated with LPS (4 nM), fMLP and PMA (1 mM each) or with PBS for control and then labelled with monoclonal antibodies against CD11b or CD14. Three different pairs of antibodies were tested as controls to verify the specificity of the energy transfer. CD11b and CD18 show a strong energy transfer on resting (ETₚ≅60%±2%) and LPS stimulated monocytes (ETₚ≅65% ±2%) and were used as positive control, while CD14 (clones Uchm1 and ×8) and CD33 showed no vicinity whatsoever and acted as negative control (ETₚ<5%). Energy transfer values are indicated in units of the Cy5 emissions, excited at 488 nm (acceptor-sensitised emission, equation 1) and differ significantly from the control with p<0,05 (*).

b. Concentration-dependent effect of LPS and ceramide concerning the co-assembly CD11b and CD14.

c. Cells were incubated with LPS (4 nM), ceramide (10 nM), LA (1 ng/ml, 100 ng/ml). LPS 4 nM)+compound-406 (400 ng/ml, c-406) and ceramide (10 nM)+c-406 (100 ng/ml) at 37° C. for 15 minutes and labelled with monoclonal antibodies. The energy transfer values differ significantly compared to incubation without the LPS antagonist at p<0,05 (*). The results represent a mean value of ±SD of 5 independent experiments.

d. CHO cells transfected with human CD14 (-unlabelled ceramide)(●)+unlabelled ceramide (∘)) or with a pPol-DHFR vector (-unlabelled ceramide (▪)+unlabelled ceramide (□)) were incubated with the specified concentrations of [¹⁴C]N-palmitoyl-D-sphingosine and the ¹⁴C bound to the cells determined. Each data point represents the mean value ± SD of three measurements. The experiment was repeated twice with comparable results.

FIG. 2

Alternative Agonists Induce the Co-assembly of CD11b and CD14

a. The effects of LPS (4 nM), lipoteichoic acid (1 mg/ml, LTA), Cer (40 nM), phosphatidyl ethanol amine (10 nM, PE), phosphatidyl inositol (10 nM, PI), phosphatidyl serine (10 nM, PS), phosphatidyl choline (10 nM, PC), lyso-phosphatidyl choline (10 nM, LPC) and sulfogalactosyl ceramide (SG-cer, 40 nM) on the co-assembly of CD11b and CD14 were analysed by means of acceptor-sensitised emission (equation 1).

b. The effects of LPS (4 nM), heat shock protein 70 (7 nM, HSP70), delipidised HSP70/7 nM, dHSP70) and SG-cer+dHSP70 (40 nM+7 nM) on the co-assembly of CD11b and CD14 were analysed by means of acceptor-sensitised emission (equation 1). They differ significantly from the control with p<0,05 (*). The data are mean values ± SD of 10 independent experiments.

FIG. 3:

Effect of Modifications in the “Raft” Structure on the CD14/CD11b Complex

a. Cells were incubated with methyl cyclodextrine/cholesterol (4.6 mg/ml 100 mg/ml, M-CD) for the cholesterol loading and propyl cyclodextrine (0.15 mg/ml, P-CD) for the cholesterol depletion at 37° C. for 30 minutes and additionally labelled with monoclonal CD14-PE, CD33-PE and CD11-b-PE antibodies. The expression density of the receptors was investigated and calculated in relation to the untreated control (100%). Differences were significant in the receptor expression vis-á-vis the control at p<0.05 (*) and of CD11b and CD14 vis-á-vis CD33 at p<0.05 ($). The data are mean values ± SD of 5 independent experiments.

b. Human monocytes were treated with methyl-cyclodextrine/cholesterol (M-CD, 1 mg/ml/22 mm/ml) and propyl cyclodextrine (0.15 mg/ml, P-CD) at 37° C. for 30 minutes and additionally incubated with LPS (4 nM, ceramide (40 nM) and PBS at 37° C. for 15 minutes. After a washing step these were labelled with the antibodies against CD11b and CD14 as described above. The energy transfer values are expressed as acceptor sensitised emission (equation 1). The energy transfer values were significant at p<0.05 (*) vis-á-vis the ex vivo control and at p<0.05 vis-á-vis stimulation with LPS ($) or ceramide (§) without further treatment. The data are mean values ± SD of five individual experiments.

FIG. 4:

Analysis of the Co-assembly of Receptors with CD14 on Individual Monocytes of Patients Suffering from Sepsis or CAD

Cells of sepsis or CAD patients which had been washed ex vivo were labelled with monoclonal antibodies.

a. Co-assembly of CD11b and CD14

b. Co-assembly of CD81 and CD14

The energy transfer values are indicated individually for more than 300 cells (sepsis) and more than 600 cells (CAD), respectively, as a one-parameter histogram obtained in accordance with equation 1.

FIG. 5: Receptor Complex Concerning Congenital Immunity on Human Monocytes

Schematic presentation of the receptor complex induced by LPS or ceramide on human monocytes. One group of surface receptors contributes to a joint signal transduction complex which plays an important role in the control of in receptor expression, growth regulation and apoptosis of an inflammatory reaction and reorganisation of the cyto skeleton.

The following examples illustrate the invention in greater detail.

### EXAMPLE 1—GENERAL PROCEDURES

(A) Blood Samples

Samples of heparinised peripheral blood were obtained from healthy male and female blood donors. As determined by relevant tests, these donors were free of diseases and infections and had not taken any medication at least during the last 14 days before the blood was drawn. Blood samples were also taken from patients suffering from sepsis, a disease of the coronary arteries (CAD) as defined by coronary angiography, either an acute myocardial infarction or angina pectoris, or an acute stroke. This was done with the consent of the patients and the local Ethical Committee (No. 00/33). The blood samples of all patients were tested for LPS in a chromogenic limulus amebocyte lysate (LAL) assay (QCL-100, Biowhittaker, Walkersville, U.S.A.), half of the sepsis patients showing a positive result while all the other samples were negative with regard to LPS.

(B) Preparation of Lipoteichoic Acid

Bacillus subtilis was grown in an 8 litre shake culture. The bacteria were broken on ice by means of ultrasound treatment (Branson “Sonifier”, Branson, Schwäbisch-Gmünd Germany) and extracted with butanol at room temperature. The aqueous phase was purified by means of chromatography based on hydrophobic interaction (HIC) on octylsepharose (Fischer, Anal. Biochem. 208 (1993), 49-56; Fischer, Anal. Biochem. 194 (1991), 353-358; Fischer, Eur. J. Biochem. 133 (1983), 523-530; Leopold and Fischer, Anal. Biochem. 201 (1992), 350-355). The fractions were screened for phosphorus-rich lipoteichoic acid by phosphomolybdenum blue assays (Schnittker et al., Biochemische Zeitschrift 2000), 167-185). All lipoteichoic acid preparations turned out to be LPS negative in the LAL assay.

(C) Delipidation of HSP70

Heat shock protein 70 (HSP70) was obtained from Stressgen (Victoria, Canada). 200 g of HSP70 were delipidated in ethanol/diethyl ether (3:1) a −20° C. over night. After a centrifugation step (15 minutes at 4000 rpm), the pellet obtained was delipidated in dicthyl ether at −20° C. for four hours. After centrifugation (15 minutes. 4000 rpm), the delipidated HSP70 (dHSP70) was dissolved in 50 nM tris-HCl. pH 7.5, 100 nM NaCl, 1 mM dithiotreitol and 0.1 mM phenylmethyl sulfonyl fluoride. HSP70 tested LPS negative in the LAL assay.

(D) Preparation of Phospholipid Liposomes

Phosphatyl ethanol amine (P), phosphatidyl choline (PC), phosphatidyl inositol (PI) and phosphatidyl serine (PS) were purchased from Sigma (Deisenhofen, Germany) as chloroform solutions. The lipids were dried in a vacuum rotary evaporator and resuspended in Dulbecco-modified phosphate buffer saline solution with Ca²⁺ and Mg²⁺ (PBS) by means of ultrasound treatment. The lipid content was determined by thin layer chromatography. All liposome preparations tested LPS negative in the LAL assay.

(E) Stimulation of Blood Samples

Aliquots (100 ml) of whole blood were incubated with the following stimulatory compounds at 37° C. for 15 minutes: LPS of Salmonella minnesota (0.1-10 nM, Mw 10 kDa), ceramide (primarily stearic acid and nervonic acid) and sulfogalactosyl ceramide (SG-cer, 40 nm), both of which tested LPS free in the LAL assay (Cer, 1 nM-5.5 mM), lysophosphatidyl choline (lyso-PC, 10 mM), phorbol-12-myristate-13-acetate (PMA, 1 mM), N-formyl-L-methionyl-L-leucyl-L-phenyl alamine (fMLP, 1 mM), PE, PC, PI and PS liposomes (10 mm) which had been prepared as described above. All reagents were obtained from Sigma. In addition, the cells were stimulated with HPS70 (7 nM), dHSP70 (7 nM) and SG-cer+dHSP70 (40 nM+7 nM). After incubation, the cells were washed with cold PBS containing 0.1% of NaN₃. The synthetic LPS antagonist lipid IVa (“compound-406”, 1-400 ng/ml) and lipid A derived from E. coli (1-100 ng/ml) were made available by courtesy of Dr. S. Kusumoto. The cells were pre-incubated with the LPS antagonist at 37° C. for 15 minutes and then stimulated with LPS at the same temperature (without washing).

(F) Binding of [¹⁴C]N-palmitoyl-d-sphingosine to CHO Cells

Cells transfected with human CD14 or the pPol-DHFR vector were grown as described recently (Stelter et al., Eur. J. Biochem. 238 (1996), 457-464). The cells were trypsinated, washed twice with PBS and then taken up in PBS containing 10% of human serum. CHO cells (4×10⁵) were pre-incubated on ice in the presence of a 50 fold excess of unlabelled N-palmitoyl-D-sphingosine (Sigma) for 15 minutes in a final volume of 100 ml. After that [¹⁴C]N-palmitoyl-D-sphingosine (American Radiolabeled Chemicals Inc., St. Louis, Mo., U.S.A.) was added and the cells incubated for another 15 minutes. In order to remove non-bound activity, the cells were washed twice and then transferred to a fresh Eppendorf vessel. After repeated washing, the pellet was dissolved in 100 ml of 1% SDS, 10 mM EDTA and then added to 3 ml of scintillation liquid. The amount of cell-bound radioactivity was determined by measuring the scintillation of the liquid.

(G) Immune Staining of Stimulated Cells

Whole blood cells which had been stimulated or washed ex vivo (200 ml) were incubated on ice for 15 minutes with saturation concentrations of monoclonal fluorochrom-conjugated or biotinylated antibodies. The monoclonal antibodies CD11a (clone 25.3.1), CD16a (clone 3G8). CD18 (clone 7E4), CD14 (clone RMO52), CD33 (clone My9-RD1). CD81 (clone Js64) were obtained as R-phyco-erythrine (R-PE) conjugates and CD25 (clone B1.49.9) in biotinylated form from Coulter/Immunotech (Krefeld, Germany). CD11b (clone d12), CD11c (clone S-HCL-3), CD36 (clone CB38), CD47 (clone B6H12), CD81 (clone JS-81) and (Cd55 (clone IA10) in biotinylated form and Streptavidin-R-PE were purchased from Becton/Dickinson/Pharningen (Heidelberg, Germany) CD18 (clone IB4) in biontinylated form and CD40 (clone EA-5) as R-PE conjugate were obtained from Alexis (Grünberg, Germany) and CD14 (clones ×8 and Uchml) and CD3 (clone Cris-7) in biotinylated form and Streptavidin-Cy5 (Sa-Cy5) from Dianova (Hamburg, Germany), CD32 (clone C1KM5) and CD64 (clone 10.1) were obtained as R-PE conjugates from Caltag (Burlingame, U.S.A.) and CD91 (clone 8G1) in biotinylated form from Proen (Heidelberg, Germany). A monoclonal anti-TLRA antibody (clone HTA125) was kindly provided by Dr. Kensuke. Since no energy transfer between the antibodies directed against the CD14/CD11a and CD14/CD11c pairs was detectable either on resting or on stimulated cells, these antibody combinations were not characterised further.

Lysis of erythrocytes and washing were carried out as described (Rothe et al., Thromb. Vasc. Biol. 16 (1996), 1437-1447). After the last washing step, the samples were divided and part of each sample incubated with a saturating amount of SA-Cy5 on ice for 15 minutes and washed before measuring.

(H) Modifications of the Raft Structure

Mononuclear cells were isolated by means of “Histopaque 1077” density gradient centrifigation (Sigma) from samples of heparinised blood. Methylised β-cyclo-dextrine (Sigma) was used as an effective donor for the cholesterol loading of the cell. Aliquots of mononuclear cells (5×10⁵ cells/ml) were resuspended in 3 ml of PBS and incubated at 37° C. for 30 minutes with complexes of cholesterol (100 mg/ml) and methlyl-β-cyclodextrine (4.6 mg/ml) which had been prepared for the cholesterol loading as described by Christian et al. in J. Lipid. Res. 38 (1997) 2264-2272). The cells were incubated with 2-hydroxy-propyl-β-cyclodextrine (Sigma 0.15 mg/ml) at 37° C. for 30 minutes for cholesterol depletion. After incubation with cyclodextrines the cells were washed, immuno-stained as described above and the expression of antigens measured by flow cytometry.

For energy transfer experiments, 200 ml aliquots of whole blood were treated with cholesterol/methyl cyclodextrine (C/C) (100 mg/ml/4.6 mg/ml) or 2-hydroxypropyl-β-cyclodextrine (0.15 mg/ml) at 37° C. for 30 minutes and then stimulated further with LPS (4 nM) or ceramide (40 nM) as described above. The energy transfer between CD14 and CD11b was measured.

(I) Measurement of FRET by Flow Cytometry

A “FACSCalibur” flow cytometer (Becton Dickinson) equipped with an air-cooled 15 mW argon laser (excitation wavelength 488 nm), a diode laser (excitation wavelength 625 nm) and standard filters [channel 1 (FITC): 530/30 nm band pass filter; channel 2 (R-PE being used as donor dye); 585/47 nm band pass filter; channel 3 (PerCP): pass having a length of >670 nm; and channel 4 (Cy5being used as recipient dye): 661/16 nm bad pass filter] was used. The Cellquest software (Becton Dickinson) was used for the measurements. The photomultiplier (PMT) voltage was adjusted for all fluorescence channels which resulted in a mean autofluorescence of the unstained leukocytes in the centre of the first of four Log orders. Measurement was conducted without compensation. 50,000 leukocytes were required in the “list” modus, forward scattering (FSC) being used as a trigger signal.

The leukocytes were first guided to an FSC vs. SSC (side scattering) dot plot as already described (Rothe et al., Thromb. Vasc. Biol. 16 (1996), 1437- 1447). A second gate based on the donor antibody expression (channel 2) was used to separate the micro-particles, debris or aggregates. The mean fluorescence intensity values were calculated for the channels 2, 3, and 4.

(J) Determination of the FRET Efficiency (Acceptor-sensitised Emission or Donor Extinction)

FRET may be used to measure distances between surface molecules (Szollosi et al., Cytometry 34 (1998), 159-179). FRET is a non-radiation based energy transfer from an excited donor fluorophor to an acceptor fluorophor via dipole/dipole interaction. The calculations were made in accordance with a simplified version of the method described by Szollosi et al., supra. The energy transfer parameter Etₚ), which is proportional to the FRET efficiency (ET), was calculated in accordance with formula (1), A being the acceptor, D the donor, FL2 the mean fluorescence in channel 2 (donor), FL3 the mean fluorescence in channel 3 and FL4 the mean fluorescence in channel 4 (acceptor) (each value having been obtained after subtraction of the autofluorescence):

$${ET}_{p} = \frac{FL3 ( D , A ) - FL2 ( D , A ) / a - FL4 ( DA ) / b}{FL3 ( D , A )} \\ a = FL2 ( A ) / FL3 ( A ) \\ b = FL4 ( D ) / FL3 ( D ) \qquad ( 1 )$$

The FRET efficiency (ET) was calculated as extinction in accordance with formula (2)

$$ET - \frac{FL2 ( D ) - FL2 ( D , A , )}{FL2 ( D )} \qquad ( 2 )$$

According to the publication by Szollosi et al. (Cytometry 5 (1984), 210-216), an ET=5% was defined as the threshold level for significant transfer efficiency in introductory experiments. This threshold level was confirmed experimentally as the detection limit proportional to the Etₚ value. In all subsequent measurements, an ET of >5% or an ETₚ of >5% was considered a positive transfer. In some measurements, there was a discrepancy between the energy transfer calculated from the donor extinction and that calculated by sensitised emission, but this may be explained by a displacement of the donor by the acceptor. Therefore, only the extinction not affected by this phenomenon is calculated (Lakowicz, in: Principles of Fluorescence Spectroscopy, 305-341; Plenum Press, New York. 2000).

(K) Measurement of the Ceramide Level in Plasma

Plasma-ceramide levels were quantified as recently described (Liebisch et al., J. Lipod. Res. 40 (1999), 1539-1546). For this purpose, plasma samples were spiked with C8 ceramide (Sigma) and extracted according to the method of Bligh and Dyer (Ca. J. Biochcm. Physiol. 37 (1959), 911-917). Quantification was made possible by a calibration curve which had been drawn by spiking with different concentrations of natural ceramide. Measurements were carried out with an “API 365 triple quadirupol” system (Perkin Elmer, Wellesley, U.S.A.) which was equipped with a turbo ion spray interface.

(L) Statistical Analyses

The data are reported as mean values ± standard deviation (SD). For a statistical comparison between control and patient groups, the Student-t test was carried out using SPSS software (SPSS, Inc., Chicago, U.S.A.). The paired Student-t test was used to compare control samples and in vitro stimulated samples.

### EXAMPLE 2—-LIGAND-INDUCED CLUSTERING OF CD11b and CD14 in “Rafts”

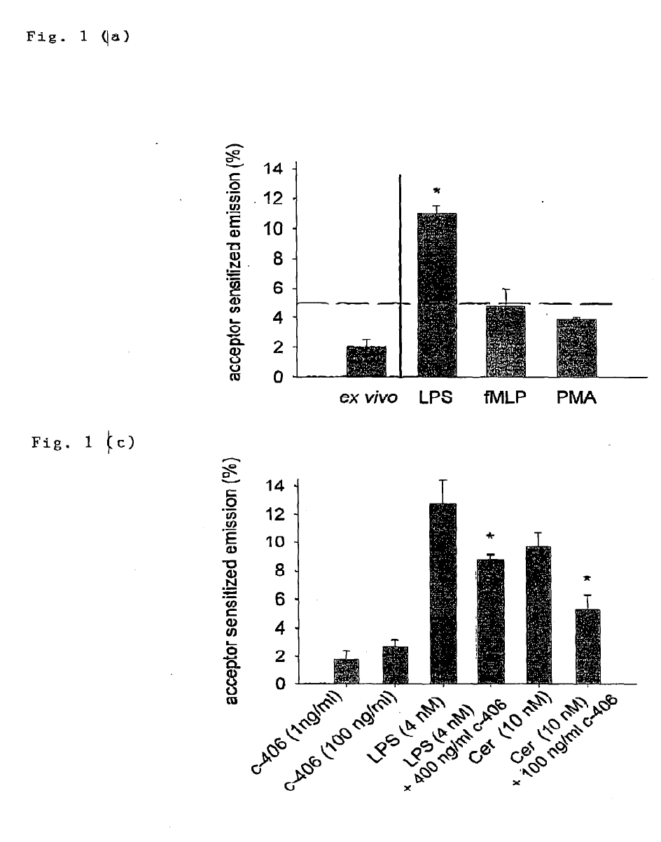

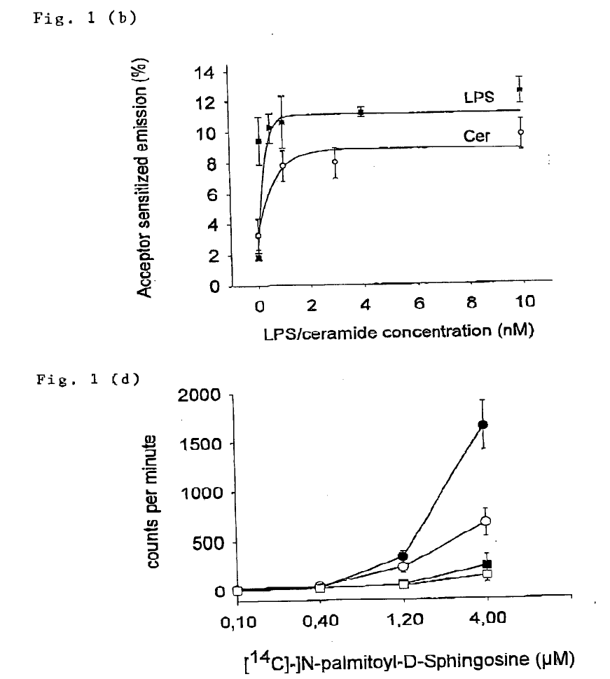

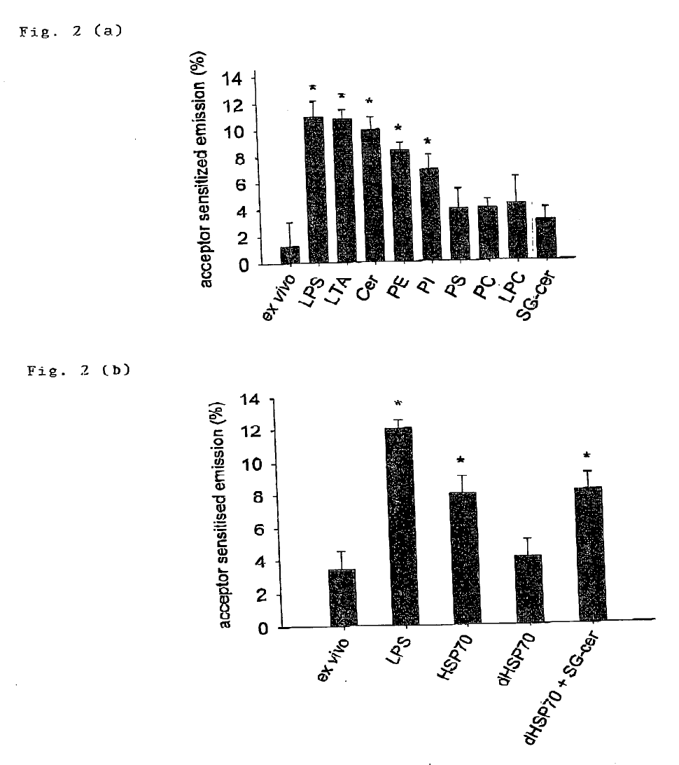

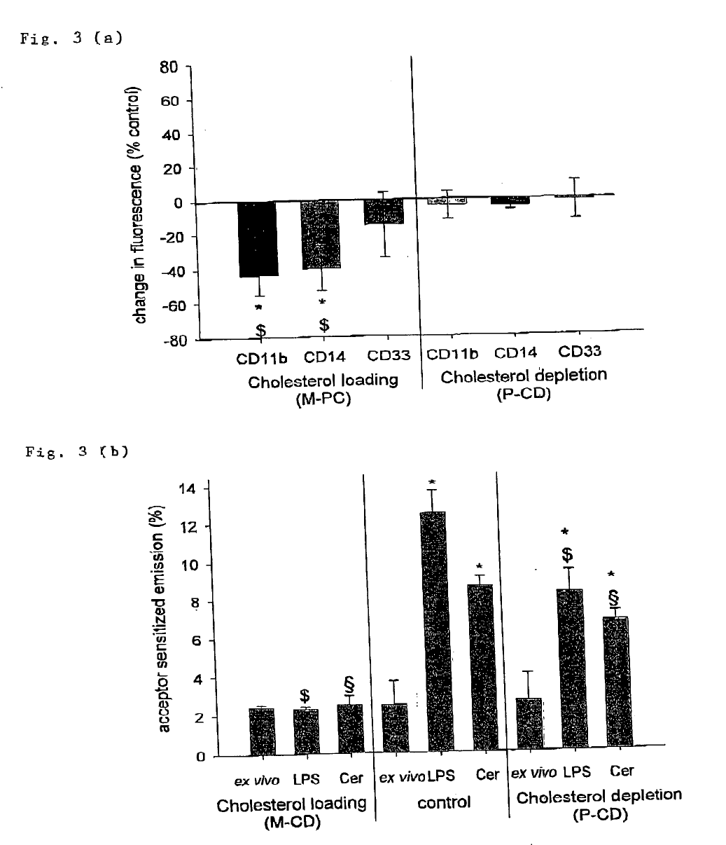

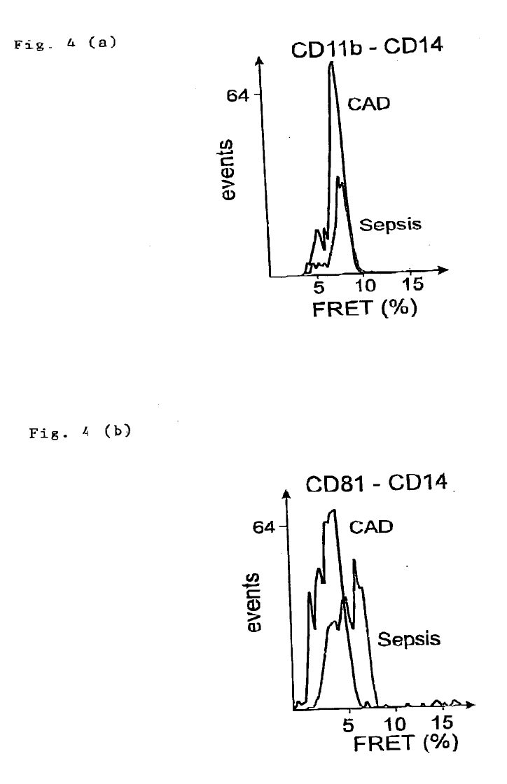

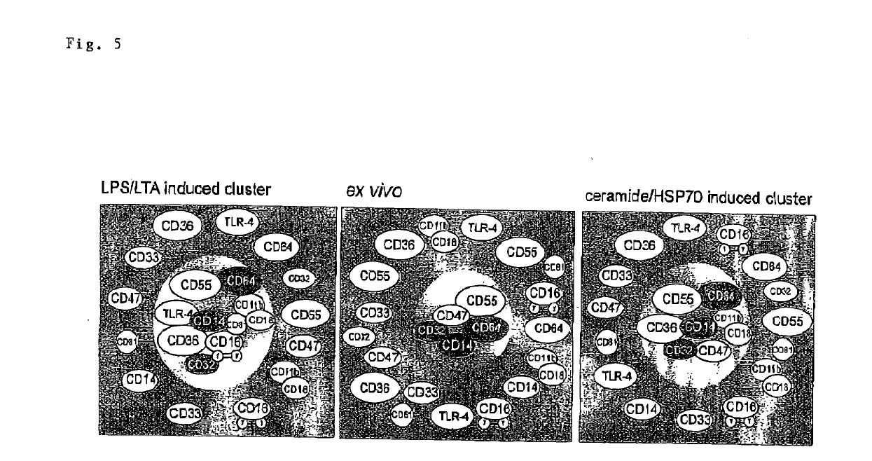

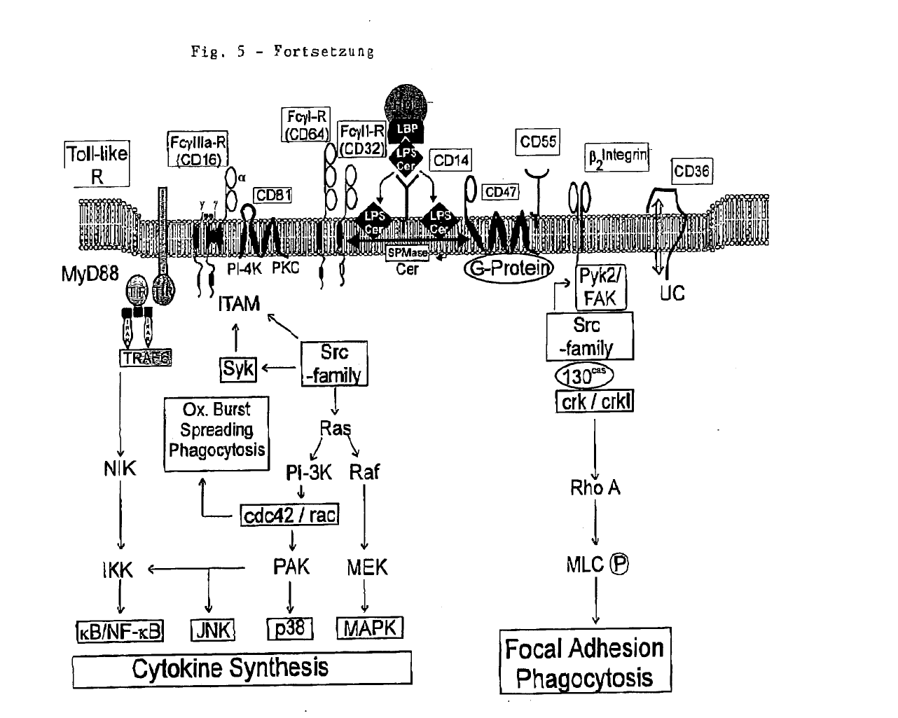

We first investigated conditions under which the co-assembly of these two receptors on monocytes is induced. Human monocytes were stimulated in vitro with LPS, fMLP and PMA and the energy transfer between monoclonal antibodies against CD11b and CD14 was measured. The results show that the co-assembly of CD14 and CD11b in a process of conformational activation is induced only LPS alone (FIG. 1a). No significant energy transfer between monoclonal antibodies against CD14 (clones ×8 or Uchm1) and CD11b was observed on resting monocytes (FIG. 1a). An energy transfer between anti-CD11b and anti-CD14 antibodies was observed on LPS-stimulated monocytes which is an indication for activation-dependent clustering of these two molecules. The effect was dose-dependent and saturation was reached at about 1 nM of LPS. Clustering was LPS specific since neither fMLP or PMA showed any effect (FIG. 1a). Similar to LPS, synthetic lipid A induced the co-assembly of CD11b and CD14.

Since it is well known that ceramide is able to imitate the LPS-induced responses, the effect of ceramide on the co-assembly of CD11b and CD14 was investigated. FIG. 1b shows the ceramide-induced energy transfer between antibodies against CD11b and CD14 (clone ×8) on human monocytes EC50_{cer} was in the range of 0.1 nM which is comparable to LPS. These results show that ceramide induces the transfer of CD14 into a receptor cluster. In order to investigate whether ceramide interacts with CD14 via a similar mechanism as LPS, a potent LPS antagonist (synthetic lipid IVa. “compound-406”) was administered before stimulating the cells with the two agonists and clustering of CD11b and CD14 then examined. The administration of “compound-406” alone (1 and 100 ng/ml) did not induce co-assembly of the receptors CD14 and CD11b (FIG. 1c), whereas administration of lipid IVa in vitro 15 minutes before stimulation reduced the LPS- or ceramide-induced co-association between CD14 and CD11b significantly (FIG. 1c).

Another indicator of the specific interaction of ceramide with CD14 was the binding of [¹⁴C]-N-palnitoyl-D-sphingosine (C16 ceramide) to CD14-transfected CHO fibroblasts which was much higher when compared to vector-transfected cells and could be inhibited by adding a 50-fold excess of unlabelled ligand (FIG. 1d). These results show that ceramide is an extracellular ligand of CD14.

For further investigation of ligands inducing the co-assembly of CD14 monocytes were incubated with lipoteichoic acid, SG-cer and different glycero-phospholipid liposomes which include PI, PS, PE, lysoPC and PC. Only lipoteichoic acid, PE and PI induced co-assembly between CD11b and CD14 (FIG. 2a). In addition, it was shown that natural HSP70 also induces clustering of CD11b and CD14, while delipidated HSP70 (dHSP70) does not induce any receptor complex. Since it is known that HSP70 binds to SG-cer, dHSP70 was recombined with SG-cer. As opposed to SG-cer on its own (FIG. 2a) and lipid-free dHSP70 (dHSP70), the SG-cer/dHSP70 complexes induced assembly of CD11b and CD14 (FIG. 2b) which is indicative of a lipid-protein-mediated rather than a protein-protein-mediated interaction of HSP70 and CD14.

It was possible to show on various cellular models that CD14 is associated with “rafts”. Therefore, the co-assembly of CD11b and CD14 was examined in dependence on the modification of the cholesterol composition of the plasma membrane with cyclodextrines in order to prove both the “raft” association and the regulatory role of the “raft” lipids with regard to the function of CD14 in human monocytes. The cholesterol loading of the membrane for 30 minutes resulted in a decrease of CD14 and CD11b expression (FIG. 3a) and inhibited the LPS-induced co-assembly of CD14 and CD11b completely (FIG. 3b). In contrast, cholesterol depletion of the membrane had no effect on the expression density of CD14 and CD11b (FIG. 3a). Co-association of both receptors after stimulation with LPS, however, was reduced significantly (FIG. 3b). Analogous measurements showed that The ceramide-induced cluster was similarly influenced (FIG. 3). This shows that an intact “raft” composition plays a regulatory role in the formation of receptor clusters.

### EXAMPLE 3—CHARACTERISATION OF SURFACE RECEPTORS VICINITY OF CD14

We investigated whether additional membrane proteins are sterically associated with CD14 after LPS or ceramide stimulation. For this purpose, we analysed the energy transfer using a series of pairs of monoclonal antibodies. It was shown that, in unstimulated cells, CD14 is clustered with the Fcγ receptors (Fcγ-R) CD32 and CD64, the complement regulating protein CD55 and the integrin-associated protein (IAP) CD47, whereas clustering with the complement-regulating proteins CD11b, CD18, the LPS signal transmitter TLR-4, the Fcγ-R CD16a, the scavenger receptor CD36 and the IAP CD81 was detectable only after LPS stimulation (Table 1a and 1b, FIG. 5). In contrast, CD47 was no longer associated with CD14 after LPS stimulation. These findings show that the LPS stimulation induces clustering of receptors on the monocyte surface, CD14, the complement-regulating proteins, TLR-4, the Fcγ receptors, CD18 and the scavenger receptor CD36 being members of these clusters LTA induces an identical cluster when compared to the LPS-induced complex.

As opposed to LPS-stimulated cells, CD16a, TLR-4 and CD81 were not associated with CD14 in ceramide-stimulated clusters, but CD47 was still associated with CD14 (table 1a and 1b, FIG. 5) Thus, the ceramide-induced cluster differs from the LPS induced one with regard to the integrated members CD14, complement-regulating proteins, CD11b, CD18, CD55, IAP CD47, Fcγ-R CD32, CD64 and the scavenger receptor CD36. SG-cer/HSP70, in turn, induces a cluster corresponding to that induced by ceramide. Therefore, LPS (LTA) and ceramide (HSP70) introduce different receptors into a cluster after binding to CD14.

TABLE 1
Co-assembly of monocyte receptors with CD:14
(acceptor-sensitised emission (%) (a) and donor extinction (%) (b))

| a | ex vivo | LPS | Cer | LTA | HSP70 |
|---|---|---|---|---|---|
| CD18(7E4)/CD14 (×8) | 2.4 ± 2.1 | 9.3 ± 1.1* | 5.4 ± 0.8* | 6.0 ± 2.1* | 6.0 ± 2.1* |
| CD36/CD14 (×8) | 3.8 ± 0.8 | 9.9 ± 0.9* | 10.1 ± 1.0* | 6.7 ± 0.9* | 8.2 ± 1.8* |
| CD55/CD14 (Uchm1) | 6.6 ± 2.5 | 8.5 ± 2.0 | 9.7 ± 1.5 | 7.2 ± 2.0 | 6.0 ± 0.5 |
| CD81 (Js64)/CD14 (×8) | 1.8 ± 0.5 | 7.3 ± 0.5* | 2.8 ± 0.7 | 6.0 ± 1.4* | 3.8 ± 2.5 |
| CD81 (JS-81)/CD14 (×8) | 2.1 ± 0.5 | 6.7 ± 0.1* | 3.1 ± 2.4 | 5.7 ± 0.1* | 1.0 ± 0.1 |
| TLR-4/CD14 (×8) | 2.2 ± 0.6 | 9.1 ± 0.6* | 3.2 ± 2.5 | 7.8 ± 2.3* | 5.4 ± 0.6 |
| CD32/CD14 (Uchm1) | 7.8 ± 1.5 | 19.6 ± 3.5 | 11.6 ± 3.4 | 6.0 ± 2.0 | 5.3 ± 2.1 |
| CD64/CD14 (Uchm1) | 9.4 ± 2.5 | 10.0 ± 2.2 | 10.4 ± 1.3 | 7.5 ± 1.8 | 10.1 ± 1.5 |
| CD47/CD14 (Uchm1) | 10.5 ± 1.0 | 4.0 ± 0.9* | 11.9 ± 3.1 | 5.8 ± 0.9* | |
| CD16/CD14 (Uchm1) | 0.5 ± 2.4 | 24.1 ± 4.2* | 3.2 ± 5.6 | 35.7 ± 3.2* | |

Cells were incubated with LPS (4 nM), ceramide (40 nM), LTA 8 (1 μg/ml) and HSP70 (7 nM) and labelled with monoclonal antibodies as described in example 1. Clones of monoclonal antibodies were selected on the basis of the highest transfer after stimulation with LPS. Energy transfer values are expressed in accordance with the emission of Cy5 (excited at 488 nm; acceptor sensitised emission, formula 1). As a control for the unspecific Fc receptor binding, the energy transfer between CD16, CD32, CD64 and two non-monocytic monoclonal antibodies (CD3 and Vβ8) or two monocytic antigens (CD25 and CD91) was measured. Stimulation of the cells with fMLP or PMA (as control) did not result in any significant energy transfer between any of the antibody pairs. The energy transfer values are expressed in accordance with the extinction of the donor fluorescence (formula 2). The energy transfer values were significant at p<0.05 (*). The data are mean values ± SD of 10 independent experiments.

### EXAMPLE 4—CO-ASSEMBLY OF MONOCYTE RECEPTORS WITH CD14 IN DISEASES ACCOMPANIED BY INFLAMMATIONS

Sepsis is often associated with the acute release of LPS and LPS-mediated cellular activation, while diseases of the coronary arteries (CAD) and stroke are diseases with a chronic inflammatory interaction between monocytes/vessel wall. In the present invention, therefore, we conducted measurements on monocytes of such patients in order to be able to investigate whether a similar co-assembly of receptors is induced in vivo. Patient samples were examined for receptor conformation on resting (ex vivo) and in vitro LPS-stimulated monocytes in comparison with blood samples of healthy donors (table 2a). The monocytes of all patient groups having diseases accompanied by inflammations, but not those of the control group showed an energy transfer between CD11b and CD14 even ex vivo before the additional in vitro stimulation. The presence or LPS in cases of sepsis could explain the co-assembly of CD11b/CD14 in this, but not in the other group of patients. Since ceramide was able to induce clustering of CD11b/CD14 in vitro, too (FIG. 1b) and earlier studies of atherogenic lipoproteins report about increased ceramide levels, the plasma ceramide level was determined by tandem mass spectroscopy. Significant changes in the plasma levels of different ceramide species (C22, C23, (24:0 and/or C24:1) were found in patients with CAD, stroke and sepsis (table 2b). However, only C24:1 was increased in all patient groups. In cases of sepsis, the greatest change with regard to the concentration of the different ceramide species was shown. The C24:1 and C22 levels were increased, while those of C24:0 and C23 were decreased.

Since ceramides might be responsible for activation of the CD14/CD11b clusters in vivo and since the formation of different clusters could be observed in vitro after induction with LPS or ceramide, respectively, the clusters were subjected to detailed examination in sepsis and CAD patients. Co-assembly of CD11b and CD14 was observed in all patient groups (FIG. 4a). Co-assembly of CD81 and CD14, however, was observed only in sepsis patients, while no energy transfer was detectable for CAD patients (FIG. 4b). These results show that, together with accompanying, ligand-dependent proteins, CD14 assembles to a specific congenital receptor complex within “rafts” and thus mediates the emission of signals during non-adaptive immune reactions.

TABLE 2
(a) Co-assmebly of CD11b and CD14, (b) plasma ceramide levels in
CAD, stroke and sepsis patients

| | Control | Sepsis | CAD | Stroke |
|---|---|---|---|---|
| | n = 20 | n = 7 | n = 20 | n = 7 |
| a) (ETₚ (%)) | | | | |
| ex vivo | 1.4 ± 0.5 | 7.7 ± 1.2* | 7.9 ± 0.6* | 13.3 ± 3.2* |
| LPS | 12.1 ± 0.5* | 9.3 ± 0.8 | 9.9 ± 0.6 | 15.2 ± 3.0 |
| b) (plasma- | | | | |
| ceramide (μM)) | | | | |
| C24:0 | 2.90 ± 0.62 | 2.1 ± 0.81* | 2.97 ± 0.83 | 3.0 ± 0.5 |
| C24:1 | 1.58 ± 0.46 | 2.98 ± 0.88* | 2.31 ± 0.64* | 2.28 ± 0.35* |
| C23 | 0.78 ± 0.19 | 0.49 ± 0.08* | 0.86 ± 0.31 | 1.02 ± 0.3* |
| C22 | 0.82 ± 0.19 | 1.06 ± 0.23* | 0.92 ± 0.24 | 0.99 ± 0.23 |
| C16 | 0.69 ± 0.61 | 1.15 ± 0.28 | 0.78 ± 0.25 | 0.72 ± 0.12 |

(a)The co-assembly of CD11b and CD14 was analysed as described in example 1 after stimulation with LPS (4 nM) and ex vivo. The energy transfer values are expressed in accordance with the emission of Cy5 (excited at 488 nm, acceptor-sensitised emisson, equation 1). The energy transfer values were significant at p < 0.05 (*)
(b)Plasma-ceramide levels were quantified by tandom mass spectroscopy and expressed accordingly as % of the control. Differences were significant at p < 0.05 (*).

(a) The co-assembly of CD11b and CD14 was analysed as described in example 1 after stimulation with LPS (4 nM) and ex vivo. The energy transfer values are expressed in accordance with the emission of Cy5 (excited at 488 nm, acceptor-sensitised emission, equation 1). The energy transfer values were significant at p<0.05 (*)

(b) Plasma-ceramide levels were quantified by tandem mass spectroscopy and expressed accordingly as % of the control. Differences were significant at p<0.05 (*).

1. A method for analysing exogenous and endogenous cell activations comprising the measurement of the assembly of receptors in a receptor cluster comprising CD14.

2. A method according to claim 1 where measurement of the assembly is carried out by means of measuring the energy transfer between the receptors.

3. A method according to claim 1 or 2 where the receptor cluster comprising CD14 also comprises one or more LPS-associated proteins, integrin-associated proteins, G-protein receptors, complement-regulating proteins, Fcγ receptors and/or scavenger receptors.

4. A method according to claim 2 or 3 where the energy transfer between an antibody against one of the proteins (receptors) according to claim 3 and an anti-CD14 antibody is measured.

5. A method according to any of the claims 2 to 4 where the energy transfer is measured by means of fluorescence resonance energy transfer (FRET).

6. A method according to any of the claims 2 to 5 where an energy transfer efficiency or an energy transfer parameter (Etₚ) of >5% are indicators of endogenous or exogenous cell activation.

7. A method for diagnosing systemic inflammations or an arteriosclerotic or inflammatory disease of the coronary arteries or cerebral arteries, respectively, or a precursor of these diseases, characterised in that the energy transfer is determined on monocytes of a patient sample by the method according to any of the claims 2 to 6, an energy transfer being indicative for a systemic inflammation of the coronary arteries or an arteriosclerotic or inflammatory disease of the cerebral or coronary arteries or a precursor of these diseases.

8. The use of a compound neutralising exogenous inflammation-promoting molecules, endogenous inflammation-mediating molecules, phospholipids, complement-regulating proteins, Fcγ receptors, scavenger receptors, integrin-associated proteins, LPS-associated receptors and/or G-protein receptors for the treatment of systemic inflammations or an arteriosclerotic or inflammatory disease of the coronary arteries or the cerebral arteries or a precursor of these diseases.

9. The use according to claim 8 where the exogenous inflammation-promoting molecules comprise LPS, lipoteichoic acid, ceramides and associated proteins, the phospholipids phosphatidyl inositol and phosphatidyl ethanol amine, the complement-regulating proteins CD11b. CD18 and CD55, the Fcγ receptors CD16a, CD32 and CD64, the scavenger receptors CD36, the integrin-associated proteins CD81 and CD47 and the LPS-associated receptors CD14 and TLR4.

10. The use according to claim 8 wherein the ceramide-neutralising compound is a C24:1 neutralising compound.

11. The use according to any of the claims 8 to 10, the neutralising compound being a specifically binding antibody or a fragment thereof.

12. The use according to any of the claims 8 to 10 where the neutralising-compound is a soluble form of a complement-regulating protein, Fcγ receptor, scavenger receptor, integrin-associated protein, LPS-associated protein and/or G-protein receptor.

13. The use according to claim 12 where the complement-regulating protein comprises CD11b, CD18 and CD55, the Fcγ receptors comprises CD16a, CD32 and CD64, the scavenger receptor comprises CD36, the integrin-associated protein comprises CD81 and CD47b and the LPS-associated receptor comprises CD14 and TLR-4.
